(19) United States
(12) Patent Application Publication
(10) Pub. No.: US 2021/0282984 A1
(43) Pub. Date: Sep. 16, 2021

(54) BABY AND ADULT DIAPERS DETECTING BACTERIAL INFECTION IN THE URINARY TRACT

(71) Applicants: ARKAN GRUP SAGLIK ÜRÜNLERi ANONiM SiRKET, Istanbul (TR); iSTANBUL ÜNIVERSITESI-CERRAHPASA REKTÖRLÜGÜ, Istanbul (TR)

(72) Inventors: Özcan ARKAN, Istanbul (TR); Özlem BALCI EKMEKÇi, Istanbul (TR); Zeynep GÜNGÖR, Istanbul (TR)

(21) Appl. No.: 17/257,486

(22) PCT Filed: Sep. 20, 2018
(86) PCT No.: PCT/TR2018/050510

§ 371 Date: Dec. 31, 2020

(30) Foreign Application Priority Data
Jul. 2, 2018 (TR) 2018/09384

(51) Int. Cl. A61F 13/84; A61F 13/49

(52) U.S. Cl. CPC A61F 13/84; A61F 2013/8497; A61F 2013/8473; A61F 13/49

(57) ABSTRACT

A diaper regularly analyzes the child's urine and changes color by detecting bacterial urinary infections and detecting urinary tract bacterial infection consisting of the layers Fluff pulp+SAP, sulfanamide, bottom cotton nonwoven, diamine and the outer textile surface.

### TECHNICAL FIELD

The invention relates to a baby and adult diaper that analyses the urine of the child and the adult, and catches the bacterial urinary infections through the solutions of sulfonamide and diamine in the diaper, and makes colour-changing urinary tract bacterial infection detection.

### BACKGROUND OF THE INVENTION

Health can be completely or partially impaired due to many reasons. These factors can be social, environmental or personal. Any factor that negatively affects the health of an individual can be described as a disease. The disease is the name given to a specific abnormal condition that occurs in the body or mind, and leads to discomfort, problem and dysfunction.

Children's diseases are basically divided into two according to their development forms; congenital and subsequently developed diseases. While the children experience their normal developmental stages respectively, they may catch diseases due to various accidents or diseases. The child is not a miniature adult, but a distinctive individual. Physiologically and mentally immature (not fully developed), but a developing organism. Due to his immaturity, he requires adult advocacy/spokesmanship to maintain and improve his health.

The urinary tract infections are the second most common type of infection observed in the children after upper respiratory tract infections. The incidence is higher in girls, except for newborns. If urinary tract infections in children are symptomatic, families go to the doctor with frequent and difficult urination and/or symptoms such as fever and abdominal pain in such patient. Although sometimes there are symptoms, families think of this situation as a simple cold, flu, etc. and expect it to pass by itself. And sometimes, asymptomatic urinary tract infection without any symptoms is experienced. The incidence of asymptomatic urinary tract infections is higher than those of symptomatic ones. In the performed researches, it is observed that asymptomatic urinary tract infections are seen in the majority of those who had symptomatic urinary tract infections.

In the current situation, since the fever is a frequent finding in children, a urine culture is requested in order to diagnose a urinary tract infection in every patient suffering from fever. And this significantly constitutes a cost for the families and the health system. The urinary tract infections that are treated late because they are noticed late, or untreated because they are not noticed cause cystitis (lower urinary tract infection) and/or pyelonephritis (upper urinary tract infection), and by the time of progress, renal scar (kidney injury) occurs. As a result, the urinary tract infections that are diagnosed and treated late cause serious problems that may lead to hypertension and kidney failure by the time of progress.

The urinary tract infections are seen in all age groups, women and men. It is known that more than 50% of all women have experienced one or more urinary tract infections throughout their lifetime. Asymptomatic urinary tract infection is frequently detected in pregnant women between a frequency of 2-7%, and this condition is determined to be associated with preterm birth and pyelonephritis. The presence of bacterial infection increases with the advancement of the age of the people. In the urinary tract infections accompanied by bacteremia, it is known that the mortality rate in elderly individuals is 5% within 28 days. Due to all these reasons, the early diagnosis and treatment of the urinary tract infections are important in terms of preventing structural and functional urinary tract abnormalities.

In the state of the art, the main function of the standard baby and adult diapers is to ensure hygiene during the toilet. The specifications that allow the baby and adult to move comfortably in compliance with their anatomical development are the arguments that are mostly commercially available and used in other functions such as sealing, quick absorption or no rash.

The invention, the baby and adult diapers detecting bacterial infection in the urinary tract, warns the users by changing the colour of the baby and adult diapers in case the users have a urinary tract infection. The nitrite is designated with the solutions applied to two layers in baby and adult diapers. It is known that the majority of the urinary tract infections seen in childhood are caused by the nitrite producing E. coli bacteria. With the present invention, it is aimed to make a quick preliminary diagnosis of bacterial urinary tract infections and to start treatment in a short time through the designation of nitrite that is accepted as a standard in the diagnosis of bacterial infection.

The present invention's, the baby and adult diapers detecting bacterial infection in the urinary tract, main purpose is to enable the users realize this from the moment the bacterial urinary tract infection first appears and to have them apply to the physician immediately. At the same time, depending on the physician's comments, it is possible to directly request a culture instead of urine analysis.

The aforementioned invention, the baby and adult diapers detecting bacterial infection in the urinary tract, eliminates the problems that will be experienced in the stages of difficulty in collecting samples from girls, who are under high risk group, in particular and rapid delivery to the hospital/laboratory for the diagnosis of urinary bacterial infection, and significantly contributes in public health.

The structural and characteristic specifications and all the advantages of the invention will be more clearly understood by the following figures and the detailed explanation written with reference to these figures, and therefore this evaluation should be carried out by taking these figures and detailed explanation into consideration.

### FIGURES HELPING TO UNDERSTAND THE INVENTION

FIG. 1: A horizontal sectional view of the baby and adult diapers detecting bacterial infection in the urinary tract

FIG. 2: A top edge sectional view of the baby and adult diapers detecting bacterial infection in the urinary tract

FIG. 3a: A view exhibiting the normal urine status of the baby and adult diapers detecting bacterial infection in the urinary tract

FIG. 3b: A view exhibiting the infectious urine status of the baby and adult diapers detecting bacterial infection in the urinary tract

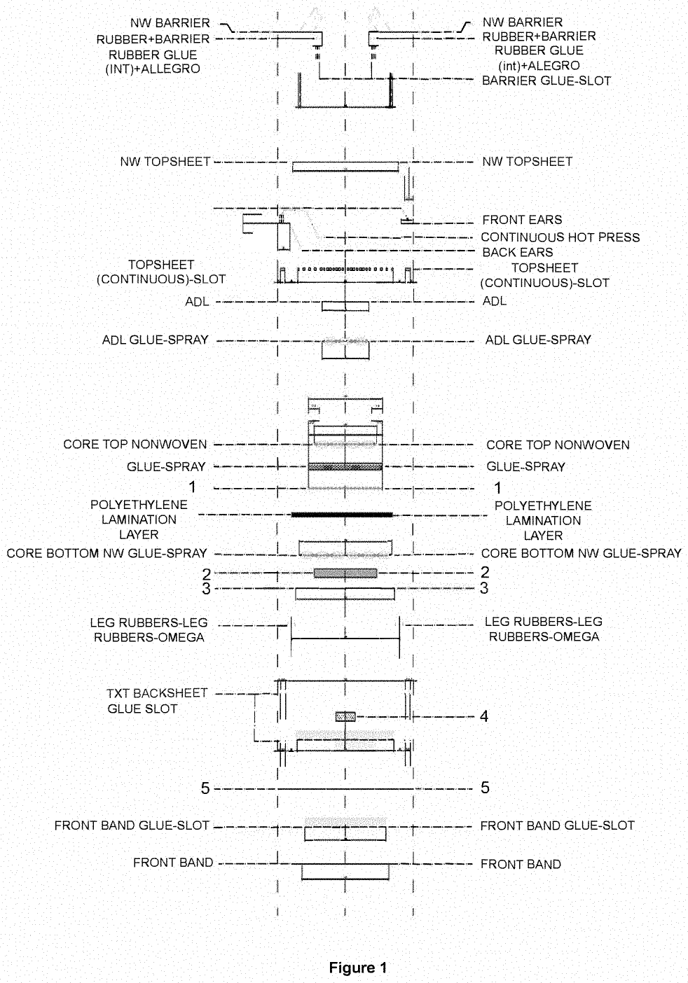

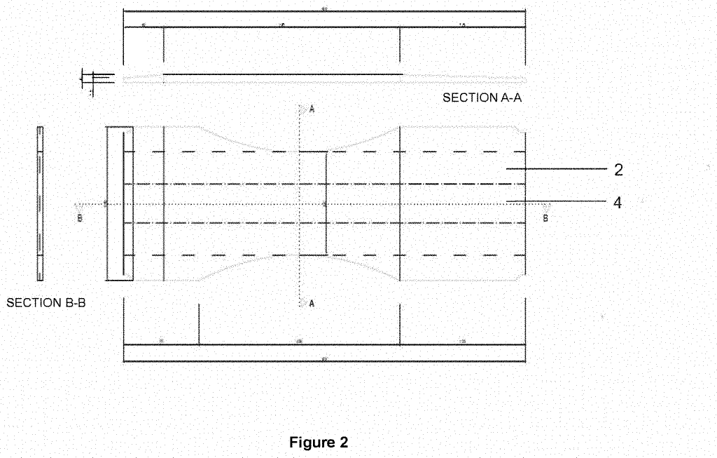

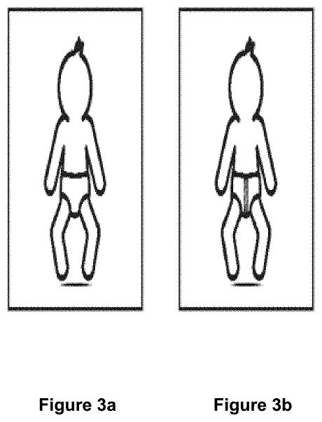

### REFERENCE NUMBERS WHICH HELPS INVENTION TO EXPLAIN

1. Fluff pulp+SAP

2. Sulfonamide

3. Lower cotton weaving

4. Diamine

5. Outer textile surface

### DESCRIPTION OF THE INVENTION

The invention, baby and adult diapers detecting bacterial infection in the urinary tract, is comprised of fluff pulp+SAP (Super Absorbent Polymer) (1), sulfonamide solution (2), lower cotton weaving (nonwoven) (3), diamine solution (4) and outer textile surface (5) parts.

According to the principle of Griess reaction, the designation of nitrite composed in urine due to bacterial infection is the most important point. The nitrite designation is carried out by means of sulfonamide (2) and diamine (4) solutions in the diaper.

In the present invention, when a person urinates, if bacterial infection is present in the urine, then the nitrite in the urine reacts with the sulfonamide (2) impregnated layer first and then with the diamine (4) impregnated layer. As a result of the reaction, a pink colour change that can be easily recognized from the outer surface of the diaper occurs. The presence of the colour change is a positive indicator in understanding the presence of infection.

In bacterial infections, particularly E. coli bacteria among the gram negative bacteria is the cause of the majority of urinary tract infections. E. coli bacteria reduces the nitrate in the urine to nitrite. The presence of nitrite in the urine indicates the presence of a bacterial infection.

The method of invention, baby and adult diapers detecting bacterial infection in the urinary tract, involves the following process steps:
Initially, to the upper layer of the lower cotton weaving (3) layer that is the lower layer of the so-called fluff pulp+SAP (1) of the baby and adult diaper, 1% chitosan containing acidic natured 1% sulfonamide (2) mixture, with a width of 3-10 cm, is applied as to be slot or spray, and the upper part of the solution is covered with polyethylene or an equal material, then reacts with the infectious urine absorbed layer,
Then, 0.8% diamine solution (4) that has been prepared in methanol is continuously applied to the inner part of the so-called outer textile surface (5) in a form of a slot with a width of 3 cm,
As a result of the last reaction with diamine (4) solution, a pink colour that indicates the presence of a bacterial infection in the urine and can be easily visible from the outermost surface of the baby and adult diaper occurs.

By means of the colour change that indicates the presence of bacterial infection, the children or adults are taken to the physician in the shortest time possible, and a more detailed examination and treatment process is started.

The technical specifications that are mentioned in each claim are followed by a reference number, and these reference numbers are used only to facilitate the understanding of the claims, therefore they should not be considered to limit the scope of any of the elements indicated by these reference numbers as they are only for illustration purposes.

It is obvious that a person skilled in the art can demonstrate the novelty revealed in the invention by using similar embodiments and/or can apply this embodiment in other areas of similar purpose used in the art. It is therefore obvious that such embodiments will not accommodate the criterion of overcoming novelty and in particular the known status of art.

1. The invention relates to a baby and adult diaper that analyses the urine of the user and detects the presence of bacterial urinary infections, as well the detection of the urinary tract bacterial infection, and it is characterized by consisting of the following process steps;
initially, to the upper layer of the lower cotton weaving layer that is the lower layer of the so-called fluff pulp+SAP of the baby and adult diaper, 1% chitosan containing acidic natured 1% sulfonamide mixture, with a width of 3-10 cm, is applied as to be slot or spray, and the upper part of the solution is covered with polyethylene or an equal material, then reacts with the infectious urine absorbed layer,
then, 0.8% diamine solution that has been prepared in methanol is continuously applied to the inner part of the so-called outer textile surface in a form of a slot with a width of 3 cm,
as a result of the last reaction with diamine solution, a pink colour that indicates the presence of a bacterial infection in the urine and can be easily visible from the outermost surface of the baby and adult diaper occurs.
